(19) United States
(12) Patent Application Publication
(10) Pub. No.: US 2007/0158241 A1
(43) Pub. Date: Jul. 12, 2007

(54) Syngas cleanup process

(76) Inventors: Juan Inga, Sapulpa, OK (US); James F. Leahy, Tulsa, OK (US)

Correspondence Address: NIXON PEABODY LLP, 161 N. CLARK STREET, 48TH FLOOR, CHICAGO, IL, 60601-3213, US

(21) Appl. No.: 11/638,259

(22) Filed: Dec. 13, 2006

Related U.S. Application Data
(60) Provisional application No. 60/750,556, filed on Dec. 15, 2005.

(51) Int. Cl. C07C 27/26; C10G 45/00

(52) U.S. Cl. 208/254 R; 518/726

(57) ABSTRACT

According to one embodiment of the present invention, a process for washing a syngas is disclosed. The process includes cooling a syngas stream to a temperature below the dew point of water. The cooled syngas stream has at least a condensed-liquid phase and a gas phase. The condensed-liquid phase contains at least one nitrogen contaminant. The process further includes separating the condensed-liquid phase of the cooled syngas stream from the gas phase of the cooled syngas stream. The process further includes removing a majority of the at least one nitrogen contaminant from the condensed-liquid phase to produce a partially-decontaminated liquid stream. The process further includes washing at least a portion of the separated gas phase of the cooled syngas stream with the partially-decontaminated liquid stream.

### CROSS-REFERENCE TO RELATED APPLICATION

This application claims the benefit of U.S. Provisional Application No. 60/750,556 filed Dec. 15, 2005, which is hereby incorporated by reference in its entirety.

### FIELD OF THE INVENTION

The invention relates to a process for removing N-containing contaminants from a synthesis gas (“syngas”). More particularly, the invention relates to a syngas cleanup process utilizing cooling of the syngas, followed by separation of condensed water and gaseous syngas.

### BACKGROUND OF THE INVENTION

Synthesis gas (“syngas”) typically contains trace nitrogen-containing compounds, principally ammonia and hydrogen cyanide. Other reactive nitrogen compound species such as cyanogen and nitrogen oxides may also be present in very small amounts. Collectively, these nitrogen-containing compounds are referred to herein as N-contaminants.

N-contaminants arise from the presence of one or more nitrogen-containing species in the feed to the syngas generator. For example, nitrogen gas (N₂) may be present in: (1) the feed natural gas; (2) the O₂ feed after air separation for an oxygen-blown syngas generation process; and/or (3) the air feed for an air-blown process. In addition to or alternatively to these sources of N₂, nitrogen-containing hydrocarbon species (especially for liquid and/or solid syngas generation feedstocks, such as residual oil or coal) may also be present in the syngas generator. The concentration of N-contaminants produced in the syngas generator may also be increased substantially through the recycle of Fischer-Tropsch tail gas into the syngas generation process. Similarly, the concentration of N-contaminants produced in the syngas generator may also be increased by recycling tail gases from other processes into the syngas generator.

Virtually all commercially practiced and proposed syngas generation processes operate at extremely high temperatures, generally in the range of about 1500° F. to about 2500° F., where the majority of the chemical reactions occur near or at chemical thermodynamic equilibrium. Under these conditions, small quantities of hydrogen cyanide (HCN) and ammonia (NH₃) are typically produced. In addition, smaller amounts of other reactive nitrogen-containing compounds such as cyanogen may also be produced. The amounts of HCN and NH₃ in a syngas depend strongly on both the nitrogen concentration in the syngas generator feed and the process conditions, particularly pressure and temperature. Prior to further processing of a syngas generator outlet stream, typical concentrations of these nitrogen-containing compounds within the outlet stream are in the range of from about 1 to about 50 vppm (volumetric parts per million) HCN and from about 5 to about 1000 vppm NH₃. Generally, the raw syngas contains between about 10 and about 30 times more NH₃ than HCN.

Removal of HCN and NH₃ from syngas is considered important because these nitrogen-containing compounds are poisons of Fischer-Tropsch catalysts, particularly non-shifting catalysts, and more particularly, those Fischer-Tropsch catalysts containing cobalt. Thus, current processes target maximum HCN and/or NH₃ concentrations lower than 100 ppb, lower than 50 ppb, and as low as 20 ppb. The lower HCN and/or NH₃ levels are required so as to achieve a Fischer-Tropsch catalyst “half-life” (the time to lose half of the initial catalyst activity) of greater than 10 days. Thus, even when the N-contaminant level is reduced, there is still some catalyst poisoning and deactivation.

Typically, a syngas stream cooler than about 200° F. is fed directly into a scrubber and the syngas stream is flushed with relatively clean water to remove the N-contaminants. The washed syngas stream generally is composed of both a gas phase and a small liquid phase. The scrubber cleans the gas phase and produces a gas stream that can be further processed. Additionally, the scrubber produces a bottoms stream containing the liquid phase of the syngas stream as well as the now contaminated water that was used to wash the syngas stream. This bottoms stream is then substantially decontaminated and fed back to the scrubber to wash another portion of the syngas stream.

It should be apparent from the above description that in classic systems, the relatively clean water fed into the scrubber not only washes the gas phase of the syngas but also contacts the liquid phase of the syngas. Because the liquid phase of the syngas contains a large amount of N-contaminants as well, the relatively clean water is quickly contaminated by the liquid phase as well as the gas phase of the syngas stream. It would be desirable to develop a system that increases the ability of the relatively clean water to wash the gas phase of the syngas stream without being saturated by N-contaminants.

### SUMMARY OF THE INVENTION

According to one embodiment of the present invention, a process for washing a syngas is disclosed. The process comprises cooling a syngas stream to a temperature below the dew point of water. The cooled syngas stream has at least a condensed-liquid phase and a gas phase. The condensed-liquid phase contains at least one nitrogen contaminant. The process further comprises separating the condensed-liquid phase of the cooled syngas stream from the gas phase of the cooled syngas stream. The process further comprises removing a majority of the at least one nitrogen contaminant from the condensed-liquid phase to produce a partially-decontaminated liquid stream. The process further comprises washing at least a portion of the separated gas phase of the cooled syngas stream with the partially-decontaminated liquid stream.

According to another embodiment of the present invention, a process for washing a syngas is disclosed. The process comprises providing a syngas stream having a plurality of phases. The process further comprises cooling the syngas stream to a temperature below the dew point of water. The cooled syngas stream has at least a condensed-liquid phase and a gas phase. The condensed-liquid phase contains at least one nitrogen contaminant. The process further comprises separating, via a liquid/gas phase separator, the condensed-liquid phase of the cooled syngas stream from the gas phase of the cooled syngas stream. The process further comprises transferring the gas phase of the cooled syngas stream to a scrubber. The process further comprises transferring the condensed-liquid phase of the cooled syngas stream including the at least one nitrogen contaminant to a stripper column. The process further comprises removing a majority of the at least one nitrogen contaminant from the condensed-liquid phase via the stripper column. The stripper column produces a bottoms stream comprising a partially-decontaminated liquid stream. The process further comprises transferring the partially-decontaminated liquid stream to the scrubber. The process further comprises washing, within the scrubber, at least a portion of the gas phase of the cooled syngas stream with the partially-decontaminated liquid stream.

The above summary of the present invention is not intended to represent each embodiment, or every aspect, of the present invention. Additional features and benefits of the present invention are apparent from the detailed description, figures, and claims set forth below.

### BRIEF DESCRIPTION OF THE DRAWINGS

FIG. 1 is a schematic of a first embodiment of the process of the invention.

FIG. 2 is a schematic of a second embodiment of the process of the invention.

FIG. 3 is a schematic of the water exchanger of the second embodiment shown in FIG. 2.

FIG. 4 is a schematic of a water exchanger and scrubber sections of a third embodiment of the invention.

FIG. 5 is a schematic of a stripper column section of a fourth embodiment of the invention.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE INVENTION

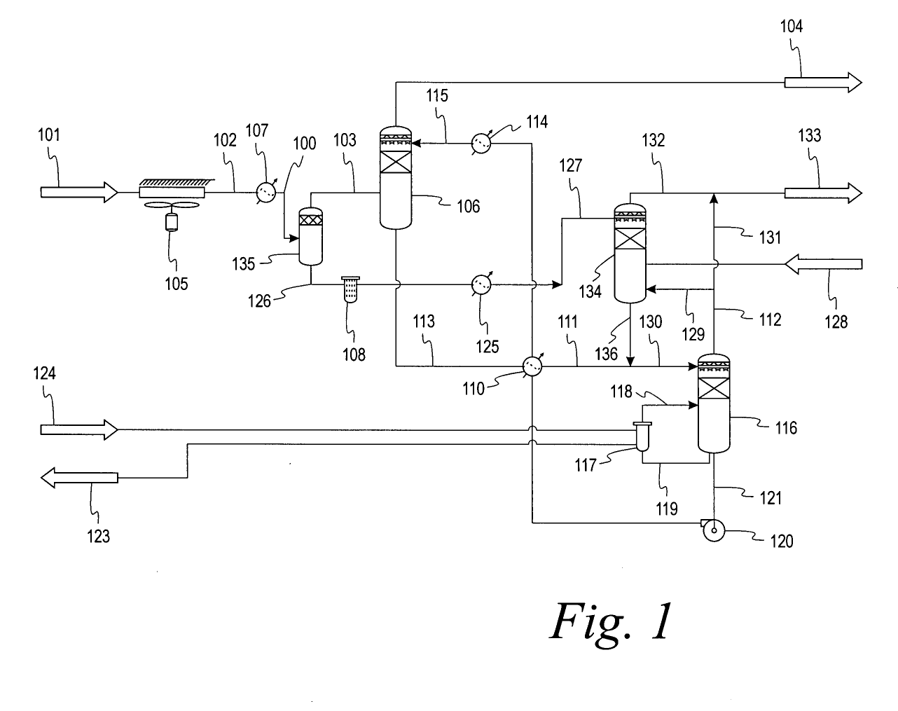

Referring to FIG. 1, a syngas stream 101 exits a syngas generation system (not shown) at temperatures generally between about 200° C. and about 315° C. after producing steam. This hot syngas stream 101 is first cooled by an aircooler 105 to a temperature generally between about 45° C. and about 75° C. A cooled syngas stream 102 exiting the aircooler 105 generally contains water or other liquid droplets that have condensed out. The cooled syngas stream 102 is further cooled by passing the cooled syngas stream 102 through a cooling water exchanger 107. The water exchanger 107 is necessary for most locations where cooling with an air cooler can only reach 48° C. Ideally, the colder a resultant further-cooled syngas stream 100, the better. The further-cooled syngas stream 100 exiting the water exchanger 107 is well below the dew point of water in syngas and the resultant condensed water droplets contain a high concentration of HCN and NH₃. The concentration of HCN and NH₃ in the condensed water droplets will vary depending upon the source and concentration of nitrogen compounds in the syngas feed. For example, for an air-fed autothermal reforming system using a pipeline quality natural gas feed, the condensed water droplets may vary between about 50 ppb (parts per billion) and about 2 ppm (parts per million) HCN and between about 2,000 ppm and about 10,000 ppm NH₃.

In some embodiments of the invention, the further-cooled syngas stream 100 is a two-phase system (e.g., liquid and gas) that is fed to a liquid/gas phase separator 135. A liquid-phase stream 126 (e.g., water) and a gas-phase stream 103 (e.g., syngas) are recovered from the liquid/gas phase separator 135. In some embodiments of the invention, the liquid/gas phase separator 135 is a knockout drum. In alternative embodiments of the invention, any vessel designed to separate liquid and gas phases may be used as the liquid/gas phase separator 135. The liquid-phase stream 126, which contains dissolved HCN and NH₃, is fed to a first stripper column 134 prior to being fed to a second stripper (not shown).

In some embodiments of the invention, the gas-phase stream 103 is washed in a scrubber 106 that contains extended-contact, solid, high-surface-area material. Acceptable extended-contact surface materials include, for example, random packing or structure packing materials that are commercially available from Cell Sime Inc., Lantec, Tricoss, or Sulzer. The scrubber 106 may be, for example, a packed column or any vessel designed to bring a gas-phase stream into contact with a liquid such as a Venturi scrubber or spray column. In addition to the gas-phase stream 103, a stripped-water stream 115 is fed to the scrubber 106. Generally, the stripped-water stream 115 is fed into a top portion of the scrubber 106. Exiting the scrubber 106 is a washed-syngas stream 104. Additionally, a water stream 113 exits the scrubber 106 and is then heated in an exchanger 110. A resulting heated-water stream 111 is then fed to a second stripper column 116.

In some embodiments, the liquid-phase stream 126 from the liquid/gas phase separator 135 is filtered by a filter device 108 prior to being heated with an exchanger 125. A number of acceptable filter devices are known to those skilled in the art and include, for example, filter bags and cartridges rated in 5 microns. The exchanger 125 may be a cooling water exchanger of the types known in the art. Exiting the exchanger 125 is a heated-water stream 127 that is fed to the first stripper column 134. In some embodiments of the invention, a steam stream 128 is directly injected into the first stripper column 134. In alternative embodiments of the invention, a second portion 129 of an offgas 112 from the second stripper column 116 is fed into the first stripper column 134 instead of the steam stream 128. In still other embodiments, both a second portion 129 of the offgas 112 and the steam stream 128 are fed into the first stripper column 134. In some embodiments of the invention, a bottoms stream 136 from the first stripper column 134 is combined with the heated-water stream 111 from the exchanger 110 to form a mixed stream 130 that is fed into the second stripper column 116. An offgas 132 may be recovered from the first stripper column 134. The offgas 132 generally contains a majority of the N-contaminants that were present in the initial syngas stream 101.

The second stripper column 116 may include a reboiler 117 fed by a second steam stream 124. The reboiler 117 may be located apart from the second stripper column 116 in some embodiments. In some embodiments of the invention, the steam streams 128 and 124 are supplied by the same source (not shown). The reboiler 117 is fed by a first portion 119 of a bottoms stream from the second stripper column 116 and produces a mixture 118 of water and water vapor. The second steam stream 124 cools as its heat energy is transferred within the reboiler 117 to the first portion 119 of the bottoms stream, resulting in a cooled-output stream 123.

A second portion 121 of the bottoms stream from the second stripper column 116 may be recycled to the scrubber 106. A pump 120 may be used to transfer the second portion 121 of the bottoms stream to the scrubber 106. In some embodiments of the invention, the second portion 121 of the bottoms stream exchanges heat with the bottoms of the scrubber 106 as a way to conserve energy prior to being injected into the scrubber 106. In some embodiments of the invention, the second portion 121 of the bottoms stream may be further cooled to a temperature between about 20° C. and about 35° C. by a water exchanger 114 prior to being injected into the scrubber 106. In fact, the colder the stream coming from the exchanger 114, the better the absorption of the N-compounds.

As discussed above, the offgas 112 exits the second stripper column 116. The second portion 129 of the offgas 112 can be fed into the first stripper column 134. A first portion 131 of the offgas 112 mixes with the offgas 132 exiting the first stripper column 132 in the vent-system stream 133.

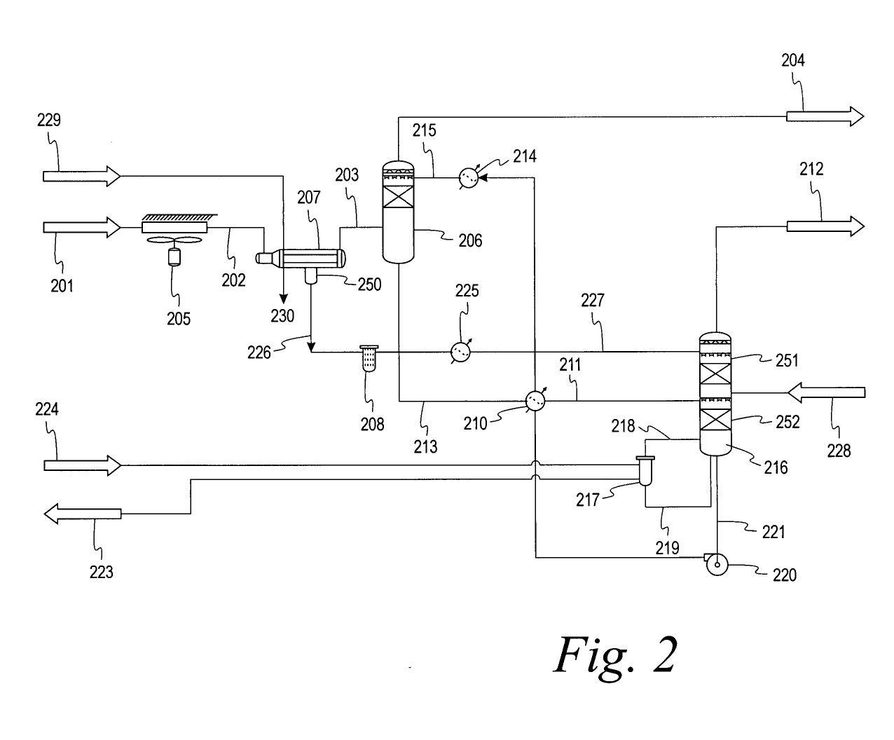

Referring now to FIG. 2, another embodiment of the process of the invention is illustrated. A syngas stream 201 from a syngas generation system (not shown) is first cooled by an aircooler 205 to a temperature between about 50° C. and about 315° C. after producing steam. In most embodiments of the invention, a cooled syngas stream 202 exiting the aircooler 205 contains water droplets. In some embodiments of the present invention, the cooled syngas stream 202 is further cooled by a cooling water exchanger 207 to a temperature between about 20° C. and about 45° C. by circulating a relatively cool-water stream 229 through the cooling-water exchanger 207. The cool-water stream 229 cools the cooled syngas stream 202 as heat is transferred to the cool water, resulting in a generally-warmer output stream 230.

The cooled syngas stream 202 is cooled to below the dew point of water in the cooling-water exchanger 207 and separation of the gaseous syngas and a condensed water phase may occur within the cooling-water exchanger 207 and may be collected in a sump 250. The cooling-water exchanger 207 is a type of exchanger having a large disengagement area so that it operates as an exchanger and a knock-out vessel combined. A condensed liquid-phase stream 226 exiting the sump 250 generally contains a high concentration of HCN and NH₃. The concentration of HCN and NH₃ in the condensed water droplets of the condensed liquid-phase stream 226 will vary depending upon the source and concentration of nitrogen compounds in the syngas stream 201. For example, for an air-fed autothermal reforming system using pipeline quality natural gas feed, the concentration of N-contaminants in the condensed water droplets may vary between about 50 ppb and about 2 ppm HCN and between about 2,000 ppm and about 10,000 ppm NH₃.

The gas-phase stream 203 exiting the cooling-water exchanger 207 may then be washed in a scrubber 206 containing extended-contact, solid, high-surface-area material, such as structured or random packing. The scrubber 206 is fed a stripped-water stream 215 for washing the gas-phase stream 203 within the scrubber 206. A resulting washed-syngas stream 204 exits the scrubber 206. Also exiting the scrubber 206 is a water stream 213 that is then heated to a temperature between about 100° C. and about 170° C. in an exchanger 210. The exchanger 210 allows the exchange of energy between process stream reducing the need of utilities. A heated-water stream 211 that exits the exchanger 210 is fed at an intermediate stage of a stripper column 216. The area of the stripper column 216 above the point of injection of the heated-water stream 211 is referred to as an upper stripper 251 while the area of the stripper column 216 below the point of injection of the heated-water stream 211 is referred to as a lower stripper 252. The upper stripper 251 and the lower stripper 252 may be either similar or dissimilar in diameter and packing material. A steam stream 228 may be injected into the upper stripper 251.

The condensed liquid-phase stream 226 from the cooling-water exchanger 207 may be filtered by a filter device 208 prior to being heated with an exchanger 225. A number of acceptable filter devices are known to those skilled in the art and include, for example, depth, cartridge, or pleated filters may be utilized that have a nominal rating of 5 microns. The exchanger 225 may be a steam heater of the types known in the art. A heated-water stream 227 exiting the exchanger 225 is fed to the upper stripper 251.

In some embodiments of the invention, the stripper column 216 includes a reboiler 217 that may be fed by a second steam stream 224. The reboiler 217 may be located outside the stripper column 216 in some embodiments. The reboiler 217 may be fed by a first portion 219 of a bottoms stream of the stripper column 216. The reboiler 217 produces a mixture 218 of water and vapor. The second steam stream 224 condenses as its heat energy is transferred within the reboiler 217 to the first portion 219 of the bottoms stream, resulting in a condensate stream 223.

A second portion 221 of the bottoms stream may be fed to the scrubber 206. In some embodiments of the invention, a pump 220 may be used to transfer the second portion 221 of the bottoms stream to scrubber 206. The second portion 221 of the bottoms stream is preferably cooled by exchanging heat with stream 213 using the exchanger 210. The exchanger 210 may be either an aircooler or water exchanger of the types known in the art. The second portion 221 of the bottoms stream may then be further cooled by a water cooler 214. An offgas 212 may be collected from the top of the stripper column 216. It will be understood that associated make-up water and purge streams may be placed where appropriate.

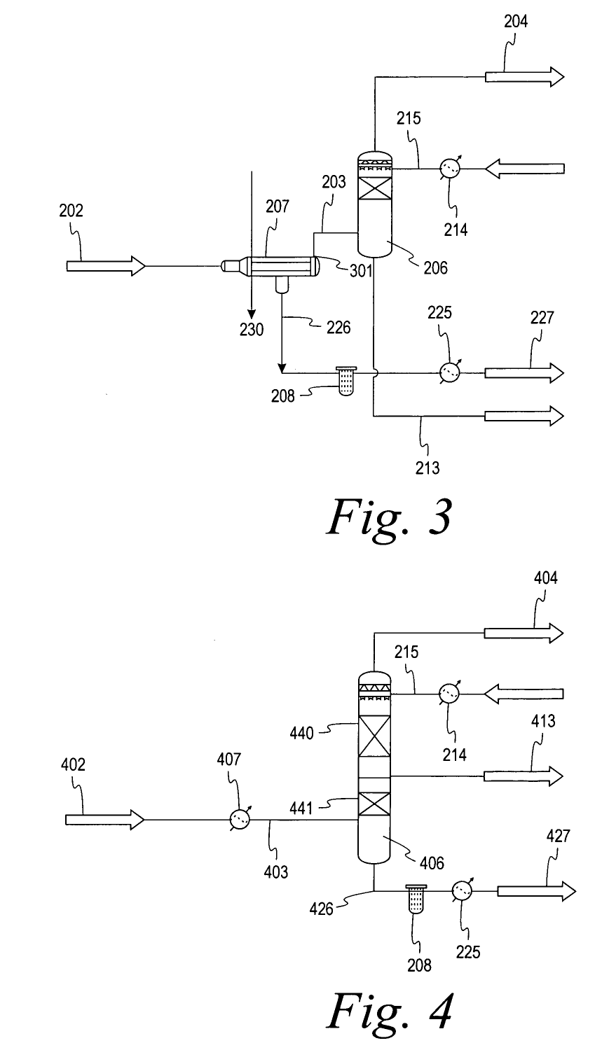

Referring now to FIG. 3, a more detailed view of the cooling-water exchanger 207 operation is illustrated. As previously described, the cooling-water exchanger 207 may be used to both further cool the cooled syngas stream 202 and to separate the resulting gas-phase stream 203 and the condensed liquid-phase stream 226. In some embodiments of the invention, the cooled syngas stream 202 is passed through the shell side of the cooling-water exchanger 207. In some embodiments of the invention, a syngas exit port 301 is provided that contains a pad to separate any water or liquid entrainment carried along with the gas-phase stream 203. These pads are also called demister pads, coalescer pads, or mist eliminators and are known to those skilled in the art. These pads may be made of, for example, polypropylene, high-density polyethylene (HDPE), nylon, stainless steel, or any other suitable material.

Yet another embodiment of the process of the invention is shown in FIG. 4. In the embodiment shown in FIG. 4, a cooling-water exchanger 407 is used to cool a syngas stream 402. The resulting condensed water droplets in a cooled syngas stream 403 are not separated in the cooling-water exchanger 407. The cooled syngas stream 403 is fed into a lower portion 441 of a scrubber 406. The scrubber 406 is partitioned by the use of structured packing into an upper scrubber 440 and the lower portion 441 of the scrubber 406. A resulting washed-syngas stream 404 exits the scrubber 406. A liquid stream 413 may be recovered from the upper portion 440 of the scrubber 406 and then fed to a stripper column such as the stripper column 216 (FIG. 2). Separation of the liquid and gas phases of the cooled syngas stream 403 occurs primarily in the lower portion 441 of the scrubber 406. The lower portion 441 of the scrubber 406 includes a coalescer pad (or another suitable pad) to separate the liquid droplets from the cooled-syngas stream 403. A separated liquid-phase stream 426 is recovered from the lower portion 441 of the scrubber 406. The liquid-phase stream 426 may be filtered by a filter device 208 prior to being heated with an exchanger 225 to produce a heated-water stream 427. It should be noted that 427 is mostly the condensed part of the cooled-syngas stream 403 since the liquid stream flowing through a pad section 440 of the scrubber 406 is collected in stream 413. In this fashion, the separation vessel such as the liquid/gas phase separator 135 (in FIG. 1) is within the scrubber 106. Stream 413 is the water sent to a stripper column (e.g., the stripper column 116 in FIG. 1). This stream does not contain significant amounts of salts. The salts are rather concentrated in the heated-water stream 427. Also, the HCN and NH₃ is concentrated in the droplets collected at the bottom of the scrubber 406 and disposed in stream 427. Only the vapor fraction of the cooled-syngas stream 403 is washed.

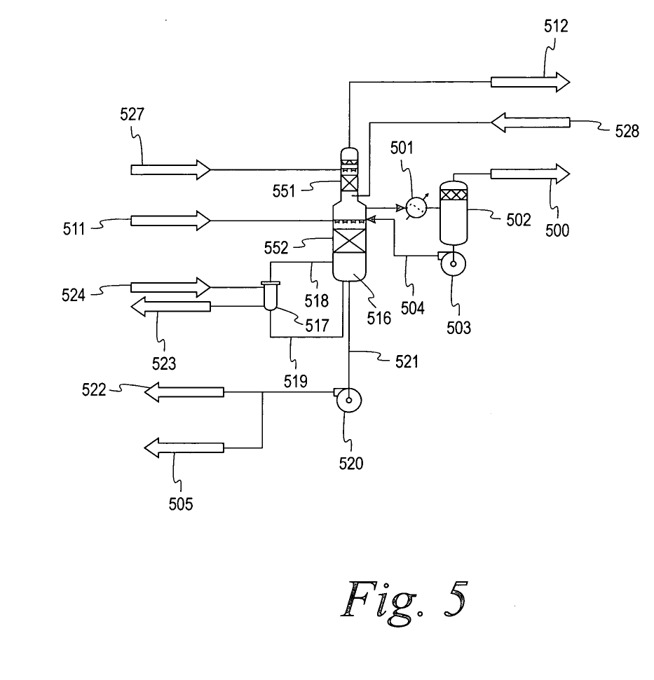

Referring also to FIG. 5, a system for recovering a liquid (e.g., water) from a liquid stream (e.g., the heated-water stream 227 in FIG. 2) is illustrated according to one embodiment of the present invention. As shown in FIG. 5, a heated-liquid stream 527 that has been separated from a cooled syngas stream (e.g., the cooled-syngas stream 202 in FIG. 2) is fed into a stripper column 516. A second heated-water stream 511 (e.g., the heated-water stream 211 that has exited the scrubber 206 in FIG. 2) is also fed into the stripper column 516. The area of the stripper column 516 above the point of injection of the second heated-water stream 511 is referred to as an upper stripper portion 551 and the area of the stripper column 516 below the point of injection of the second heated-water stream 511 is referred to as a lower stripper portion 552. The upper stripper portion 551 and the lower stripper portion 552 may be either similar or dissimilar in diameter and packing material. A steam stream 528 may be injected into the upper stripper portion 551.

A reboiler 517 is fed by a second steam stream 524. The reboiler 517 may be located apart from the stripper column 516 in some embodiments. In some embodiments of the invention, the steam streams 528 and 524 are supplied by the same source (not shown). The reboiler 517 is fed by a first portion 519 of a bottoms stream from the stripper column 516 and produces a mixture 518 of water and water vapor. The second steam stream 524 cools as its heat energy is transferred within the reboiler 517 to the first portion of the bottoms stream 519, resulting in a cooled or condensed output stream 523.

In the embodiment shown in FIG. 5, the upper stripper 551 has a smaller diameter than the lower stripper portion 552. A knockout drum 502 is provided in fluid communication with the upper stripper portion 551 of the stripper column 516. A cooler 501 is located between the upper stripper portion 551 and the knockout drum 502. Any vapors produced in the lower stripper portion 552 via the reboiler 517 can be partially condensed by the combination of the cooler 501 and the knockout drum 502. In some embodiments of the invention, a pump 503 is used to recycle a condensed water stream 504 from the knockout drum 502 back into the stripper column 516. An offgas 500 from the knockout drum 502 may also be recovered or removed. Recycling the condensed water stream 504 into the stripper column 516 increases the water flow in the stripper column 516 and results in a net-flow increase to the bottoms stream 521.

A pump 520 is provided to transfer the bottoms stream 521 to other parts of the system. For example, a first portion 505 of the bottoms stream 521 from stripper column 516 may be diverted to a water recovery process including, for example, a reverse-osmosis unit, while a second portion 522 of the bottoms stream 521 may be directed to an additional stripper, a scrubber, another component of the system, and/or out of the system entirely.

It should be noted that the partial condensation of a vapor stream from the upper stripper portion 551 of the stripper column 516 infers that the condensed water 504 may have some N-contaminants therein. However, the upper stripper portion 551 of the stripper column 516, in fact, removes most of the N-contaminants resulting in the concentration of the N-contaminants in the condensed water 504 being minimal.

In addition to the advantages and features of the invention apparent from the above description of embodiments of the invention, the invention provides other advantages. For example, the scrubber encounters significantly reduced quantities of N-contaminants than in current processes due to the separation and stripping of the N-contaminants in the liquid-phase stream prior to directing the liquid to the scrubber. Also, a significant reduction in the size of the stripper (diameter and number of trays) is possible because most of the N-contaminants are removed prior to the mixing between the water streams. This reduction of column size reduces the water required by the process. It is also envisioned that the water in the scrubber/stripper system will contain less N-contaminants.

While the present invention has been described with reference to one or more particular embodiments, those skilled in the art will recognize that many changes may be made thereto without departing from the spirit and scope of the present invention. Each of these embodiments and obvious variations thereof is contemplated as falling within the scope of the claimed invention, which is set forth in the following claims.

1. A process for washing a syngas comprising:
cooling a syngas stream to a temperature below the dew point of water, the cooled syngas stream having at least a condensed-liquid phase and a gas phase, the condensed-liquid phase containing at least one nitrogen contaminant;
separating the condensed-liquid phase of the cooled syngas stream from the gas phase of the cooled syngas stream;
removing a majority of the at least one nitrogen contaminant from the condensed-liquid phase to produce a partially-decontaminated liquid stream; and
washing at least a portion of the separated gas phase of the cooled syngas stream with the partially-decontaminated liquid stream.

2. The process of claim 1, wherein a knockout drum is used to separate the condensed-liquid phase of the cooled syngas stream from the gas phase of the cooled syngas stream.

3. The process of claim 1, wherein the condensed-liquid phase is fed to a first stripper, the first stripper being adapted to remove at least a portion of the at least one nitrogen contaminant.

4. The process of claim 3, wherein the first stripper includes at least an upper portion and a lower portion.

5. The process of claim 4, wherein the upper portion of the first stripper is smaller in diameter than the lower portion of the first stripper.

6. The process of claim 3, wherein the condensed-liquid phase is fed to a second stripper after at least a portion of the at least one nitrogen contaminant has been removed by the first stripper, the second stripper being adapted to remove at least a second portion of the at least one nitrogen contaminant, the first and second strippers being adapted to remove the majority of the at least one nitrogen contaminant from the condensed-liquid phase.

7. The process of claim 3, wherein the condensed-liquid phase is run through a filter prior to being fed to the first stripper.

8. The process of claim 7, wherein the filter is adapted to filter out particulates in excess of 5 microns in size.

9. The process of claim 1 further comprising cooling the partially-decontaminated liquid stream prior to washing the portion of the separated gas phase of the cooled syngas stream with the partially-decontaminated liquid stream.

10. The process of claim 9, wherein the partially-decontaminated liquid stream is cooled to less that 35° C.

11. The process of claim 1, wherein the cooling of the syngas stream and the separating of the condensed-liquid phase are performed by a water exchanger having a large disengagement area.

12. The process of claim 11 further comprising collecting the separated condensed-liquid phase in a sump attached to the water exchanger.

13. The process of claim 1, wherein the separating of the condensed-liquid phase from the gas phase occurs within a scrubber having a lower portion, the separation of the condensed-liquid phase occurring within the lower portion of the scrubber.

14. The process of claim 13, wherein the scrubber includes a coalescer pad adapted to separate the condensed-liquid phase from the gas phase.

15. The process of claim 13, wherein the washing of the portion of the separated gas phase with the partially-decontaminated liquid stream occurs within the scrubber.

16. A process for washing a syngas comprising:
providing a syngas stream having a plurality of phases;
cooling the syngas stream to a temperature below the dew point of water, the cooled syngas stream having at least a condensed-liquid phase and a gas phase, the condensed-liquid phase containing at least one nitrogen contaminant;
separating, via a liquid/gas phase separator, the condensed-liquid phase of the cooled syngas stream from the gas phase of the cooled syngas stream;
transferring the gas phase of the cooled syngas stream to a scrubber;
transferring the condensed-liquid phase of the cooled syngas stream including the at least one nitrogen contaminant to a stripper column;
removing a majority of the at least one nitrogen contaminant from the condensed-liquid phase via the stripper column, the stripper column producing a bottoms stream comprising a partially-decontaminated liquid stream;
transferring the partially-decontaminated liquid stream to the scrubber; and
washing, within the scrubber, at least a portion of the gas phase of the cooled syngas stream with the partially-decontaminated liquid stream.

17. The process of claim 16 further comprising cooling the partially-decontaminated liquid stream prior to washing the portion of the separated gas phase of the cooled syngas stream with the partially-decontaminated liquid stream.

18. The process of claim 17, wherein the partially-decontaminated liquid stream is cooled to less that 35° C.

19. The process of claim 16 further comprising filtering the condensed-liquid phase prior to transferring the condensed-liquid phase to the stripper column.
